(19) United States
(12) Patent Application Publication
(10) Pub. No.: US 2016/0356511 A1
(43) Pub. Date: Dec. 8, 2016

(54) METHODS AND APPARATUS FOR COMBATING SICK BUILDING SYNDROME

(71) Applicant: Philadelphia University, Philadelphia, PA (US)

(72) Inventors: Alexander A. Messinger, Ardmore, PA (US); Diana R. Cundell, Philadelphia, PA (US); Brian R. George, Pottstown, PA (US)

(21) Appl. No.: 14/986,965

(22) Filed: Jan. 4, 2016

Related U.S. Application Data
(63) Continuation of application No. 12/705,843, filed on Feb. 15, 2010, now abandoned.

(51) Int. Cl. F24F 3/16; A61L 9/00; F24F 13/02

(52) U.S. Cl. CPC F24F 3/16; F24F 13/0254; A61L 9/00; F24F 2003/1675; F24F 2221/20

(57) ABSTRACT

Methods and apparatus for combating sick building syndrome include a plenum that is at least partially bounded by fabric comprising at least one naturally occurring botanically based, antimicrobial, morbidity-inducing fabric and preferably a fan for introducing air into the plenum for passage outwardly through the fabric thereby to cleanse the air of microbes contributing to sick building syndrome.

### CROSS-REFERENCE TO RELATED PATENT APPLICATIONS

This patent application is a 35 USC 120 continuation application of pending U.S. utility patent application Ser. No. 12/705,843 entitled “Methods and Apparatus for Combating Sick Building Syndrome, filed Feb. 15, 2010.

### BACKGROUND OF THE INVENTION

Field of the Invention

This invention relates to maintenance and improvements of internal environments within buildings and more specifically relates to methods and apparatus for combating “Sick Building Syndrome” (“SBS”).

Description of the Sick Building Syndrome Problem and Prior Art Approaches to the Problem

Current design of buildings seeks to maximize energy efficiency and comfort for the building inhabitants using centralized heating and cooling systems. As a result, buildings now being built are becoming increasingly airtight, relative to buildings of prior years. Combined with the use of inexpensive building materials such as particle board, drywall and acoustical tile used as ceiling tiles, the modern design and construction approach has fostered a series of ailments affecting people living and working in these building environments. These ailments have been collectively termed Sick Building Syndrome (SBS).

Sick buildings are characterized by poor air circulation and imbalance in humidity which together allow build-up of biological and chemical contaminants.

The adverse impact, both economically and on public health, is significant: The United States Environmental Protection Agency has estimated that $61 billion dollars are lost in medical costs and worker absenteeism annually.

It is further estimated that if ventilation and lighting were improved in commercial buildings in the United States, there would be somewhere between 16 million and 37 million fewer cases of influenza or the common cold each year, and an 8% to 25% decrease in symptoms for the 53 million persons that suffer from allergies and the 16 million persons who are asthmatic in the United States, and further that there would be a 20% to 50% reduction in so-called “Sick Building Syndrome Health Symptoms”.

SBS health symptoms have been reported to be most prevalent in persons suffering from allergies and asthmatics, whose sensitivity is often greatest to even low levels of indoor airborne biological contaminants that include microbes, especially molds. Since allergies are estimated to affect at least 1 person in every 6 of the population of the United States, build-up of these indoor contaminants is clearly of great concern.

While studies have shown that locations with SBS may have high levels of both airborne molds and bacteria, most researchers have devoted their efforts to the study of the molds and their effects due to the ease of identification of molds, the dramatic levels of spore release and responsiveness of molds to remediation by increasing air flow and decreasing humidity. Spore release by molds can amount to as high as approximately 300,000 spores per gram of mold mass.

The current consensus among environmental microbiologists is that elevated levels of at least 3 genera of airborne molds, namely penicillium, aspergillus and alternaria, can produce symptoms of SBS. These species, together with cladosporium, are believed to constitute greater than 90% of the viable mold flora in ambient air in a variety of environments, with up to a 50% increase in airborne alternaria and cladosporium occurring in Fall and Winter.

Elevated levels of airborne staphylococci as well as aerosolized water contaminated by legionella or gram-negative bacteria and their products have also been linked with SBS. Collectively, these bacteria comprise the dominant species in ambient air and are important agents for a wide range of infectious respiratory, gastro-intestinal and cutaneous human diseases.

Products currently on the market and promoted to remove such airborne contaminants are primarily focused against allergens and work to trap them in electrostatically-charged filters, which require periodic replacement or cleaning.

### SUMMARY OF THE INVENTION

In one of its aspects, this invention provides a series of unique modular, adaptable and esthetically pleasing units for retrofitting tightly sealed buildings to combat Sick Building Syndrome (SBS). The units utilize fabric to capture airborne molds and bacteria responsible for SBS and, in some embodiments, also work to diffuse and balance indoor light levels. Measured reductions in indoor mold levels from ambient air using modular units according to the invention within a one month operational period ranged from 71% to 83%.

In another one of its aspects, as illustrated generally in FIG. 1, this invention provides a preferably modular unit for improving indoor air quality where the unit includes a frame surrounding an open interior and defining an outer periphery of the unit, air permeable, preferably naturally occurring botanically based antimicrobial, morbidity-inducing fabric secured to the frame periphery on a first side of the frame and covering the open interior of the first side of the frame, an air impermeable member secured about the frame periphery on the remaining side of the frame and covering the frame periphery on the remaining side, at least one aperture preferably formed in the frame and being adapted to house a fan therein with an optional second aperture preferably being provided for a second fan or for air bleed, and at least one fan housed in the aperture for blowing air from outside the frame into the frame interior, for subsequent passage of blown into the frame interior outwardly through the fabric.

The fabric is preferably secured to the frame by Velcro; the air impermeable member is preferably fiberboard; the frame preferably has four members, two of which are preferably spaced apart lateral members, with the remaining two being bottom and top members; the unit preferably further includes a bracing member extending between and affixed to both the bottom and top members; the frame is preferably rectangular.

In the unit illustrated generally in FIG. 2, the unit bottom and top members have edges preferably defining top and bottom portions of the first side of the frame periphery that are curved; the curved edges are preferably in the form of an S-curve; the curved edges of the top and bottom members preferably are tangent to the upstanding members at juncture therewith, and further preferably the edges of the top and bottom members are parallel. The fabric may be curved along the vertical length of the fabric in the same shape as the edges of the top and bottom portions of the first side of the frame periphery. The edges of the bottom and top members are preferably parallel. Outwardly facing surfaces of the upstanding lateral members are preferably parallel to facingly contact preferably upstanding lateral members of adjacent units. Positioning the fabric in the curved shape shown permits a greater area of fabric per unit area occupied by the unite than the purely “rectangularly configured” unit illustrated in FIG. 1.

In another one of its aspects, as illustrated generally in FIG. 3, the invention provides a preferably modular unit for improving indoor air quality where the unit includes a frame having an open center and defining the outer periphery of the unit, with the frame including a pair of parallel, spaced apart, lateral members connected by tensioning cables running from upper extremities of respective lateral members to lower extremities of respective opposed lateral members, and at least one aperture in at least one of the lateral members preferably opening in a direction perpendicular to the open center. The unit further preferably includes air permeable, preferably naturally occurring botanically based antimicrobial morbidity-inducing fabric secured about the frame periphery on a first side of the frame and covering the open center. The frame periphery on the remaining side of the frame is preferably planar and adapted to fit against a vertical wall. The unit includes a fan mounted in the aperture for blowing air from outside the frame into the frame interior for subsequent air passage from the frame interior outwardly through the fabric. The tensioning cables are adapted preferably to connect to one or more cable gripping devices preferably mounted in or on the vertical wall so that upon connection to the cable gripping devices, the tensioning cables preferably pull the planar remaining side of the frame against the vertical wall thereby closing the unit interior so that air may enter the unit only by action of the fan.

In still another one of its aspects, as illustrated generally in FIG. 4, this invention provides a preferably modular unit for improving indoor air quality where the unit includes a frame surrounding an open interior and defining the outer periphery of the unit. Air permeable, preferably naturally occurring botanically based antimicrobial, morbidity-inducing fabric is secured about the frame periphery on a first side of the frame and covers the open interior on the first side of the frame. At least one aperture is formed in the frame. A fan is preferably mounted in the aperture and serves to blow air from outside the frame into the frame interior for subsequent air passage from the frame interior outwardly through the fabric with an optional second aperture, if provided, being a bleed air escape.

The unit is preferably mountable against a vertical wall by an upper portion of the frame preferably hanging on at least one member protruding from the wall with a second side of the frame being substantially flush with the wall so that air blown into the open interior by the fan escapes from the frame interior by passing through the fabric. The air permeable, preferably naturally occurring botanically based fabric preferably comprises multiple fabric layers and is preferably secured to the frame by Velcro. The frame preferably has four members with two of those members being spaced apart upstanding lateral members and the remaining members being bottom and top members. The frame is preferably rectangular, with the bottom and top frame members having edges, preferably defining top and bottom portions of the first side of the frame periphery, that may be curved. When curved, the edges are preferably in the form of an “S-curve”; the curved edges are preferably tangent to the upstanding members at juncture therewith, and edges of the bottom and top members are preferably parallel, all as shown generally in FIG. 4. The curved edges allowed greater area of fabric than a unit with straight edges, as described above for the unit of FIG. 2 vis-à-vis the unit of FIG. 1.

In still yet another aspect of the invention, as illustrated generally in FIG. 5, there is provided a preferably modular unit for improving indoor air quality where the unit includes a frame surrounding an open interior and defining the outer periphery of the unit with air permeable, preferably naturally occurring botanically based antimicrobial, morbidity-inducing fabric secured about the frame periphery on first and second sides of the frame and covering the open interior on the first and second sides of the frame. A pair of apertures is preferably formed in the frame, one aperture in the upper extremity of the frame and the other aperture in the lower extremity of the frame. At least one fan is mounted on the frame and further is preferably aligned with one of the apertures, for drawing air from outside the frame into the frame interior through at least one of the aligned apertures for subsequent air passage from the frame interior outwardly through one of the two sections of fabric. The unit is preferably hung from the ceiling or other structure located well above the floor of a room; the unit may also sit on the floor. Air drawn into the open interior of the frame by the fan escapes from the frame interior by passing through one of the two sections of the fabric. The sections of air permeable fabric may comprise multiple fabric layers, preferably secured to the frame by Velcro as illustrated generally in FIG. 6. Alternatively, the rear or second side of the frame may be closed, a per the unit illustrated in FIG. 1. In either case, the frame preferably has four members with two of those being upstanding spaced-apart lateral members and the remaining members being bottom and top members. The frame is further preferably rectangular. The top and bottom members of the frame are preferably parallel. The unit may further include a brace extending between the upstanding spaced-apart lateral members proximate the vertical midpoints thereof. The outwardly facing lateral surfaces of the frame are preferably planar to facingly contact an outwardly facing lateral surface of the frame of an adjacent unit, preferably along the entire vertical length of the respective lateral surfaces.

In still another one of its aspects, as illustrated generally in FIG. 7, this invention provides a modular unit for improving indoor air quality where the unit includes a horizontal frame surrounding an open interior and defining a portion of the outer periphery of a filter portion of the unit. Air permeable, preferably naturally occurring botanically based antimicrobial, morbidity-inducing fabric is secured around the frame periphery on a downwardly facing side of the frame and covers the open interior of the downwardly facing side of the frame. Porous light-reflective fabric is preferably secured about the frame periphery on the upwardly facing side and covers the frame periphery on the upwardly facing side of the frame. At least one fan is mounted in the frame for blowing air from outside the frame into the frame interior for subsequent passage of air blown into the frame interior outwardly through the air permeable preferably naturally occurring botanically based fabric. An exterior portion of the frame outer periphery is preferably planar for facingly contacting a portion of a window. The unit further preferably includes at least one cable-like member extending upwardly from the frame for upwardly supporting the frame about a pivotal hook-and-eye connection at a position remote from the planar portion of the frame, preferably to permit weight of the frame to urge the frame against the window and maintain the frame in a horizontal plane.

The unit further optionally includes at least one solar cell positionable to receive solar energy through the window, connected to and powering the fan. The air permeable fabric is preferably secured to the frame by Velcro; the frame preferably has four members; the frame is preferably rectangular; the vertically supporting member is preferably a cable and the downwardly facing surfaces of portions of the frame that are parallel to the window are preferably parallel with one another. Optimally, multiple layers of the antimicrobial fabric, constructed to be easily removable and in the form of replaceable modular assemblies, as illustrated generally in FIG. 8, may also be used.

In still another one of its aspects, as illustrated generally in FIG. 10, this invention provides a preferably modular unit for improving indoor air quality where the unit includes a frame surrounding an open interior and defining the outer periphery of the unit, air permeable preferably naturally occurring botanically based antimicrobial, morbidity-inducing fabric secured about the frame periphery on a lower side of the frame and covering the open interior on the lower side of the frame, and air porous preferably reflective preferably non-woven fabric secured about the frame periphery on the remaining upper side of the frame and covering the frame periphery on that remaining upper side. The unit has at least one aperture formed in the frame with the aperture adapted to house a fan therein. A fan is preferably mounted in the aperture for blowing air into the frame interior for subsequent passage of that air outwardly through the air permeable fabric. The unit further includes a pair of struts extending vertically from the frame for supporting the unit in mid-air within a room in a building. The fabric is preferably secured to the frame by Velcro; the frame is preferably rectangular and has four members, two being longer than the others; as illustrated in FIG. 10, the unit preferably includes bracing cables within the frame interior extending between and affixed to respective ones of the longer members of the frame. In the unit, two parallel sides of the frame each have bottom edges that are parallel one with another. The lower edges of the frame may be gently curved, as shown in FIG. 11, to enhance the aesthetics of the unit and to provide a larger area of the air permeable preferably naturally occurring botanically based antimicrobial, morbidity-inducing fabric for a given area occupied by the unit.

In still another one of its many aspects, as illustrated generally in FIG. 12, the invention provides passive apparatus for improving indoor air quality, where the apparatus includes framework comprising a plurality of vertically upstanding members positioned at the corners of imaginary contiguous rectangles, one continuous edge of the contiguous rectangles being considered the rectangle fronts. Each of two pairs of the vertically extending members are associated with at least one of the rectangles. For each rectangle, one pair of members has a first one of its members at the right front of the rectangle and the second one of its members at the right rear of the rectangle, and a second pair of members has a first one of its members at the left front of the rectangle and a second one of its members at the left rear of the rectangle.

First and second horizontally oriented support members are preferably associated with respective ones of the rectangles and are preferably positionable on a floor or other surface to provide vertical support for the apparatus. These support members respectively preferably extend transversely between lower extremities of the first and second members of the respective pairs of upstanding members associated with the respective rectangle.

A plurality of vertically spaced apart pairs of parallel bracing members associated with respective ones of the rectangles connect respective ones of the upstanding first and second members of the respective pairs of upstanding member and lie along respective sides of the rectangle extending transversely away from the front. Uppermost lateral members associated with respective ones of the rectangles extend between and preferably slidably engage uppermost correspondingly positioned pairs of the horizontally extending parallel bracing members associated with respective rectangle.

Pluralities of lower lateral members below the uppermost lateral members associated with respective ones of the rectangles extend between and preferably slideably engage vertically corresponding positioned pairs of the preferably horizontally extending preferably parallel bracing members associated with the respective rectangle.

Air permeable, preferably naturally occurring botanically based antimicrobial, morbidity-inducing fabric is connected to at least one of the uppermost lateral members and extends downwardly always along and preferably between at least the vertically adjacent pairs of lower lateral members associated with respective rectangle(s). The lateral members are preferably movably positionable along the parallel bracing members, between front and rear, to cause fabric connected thereto and extending therefrom to conform to selected contours, with at least a portion of such contour preferably approximating the shape of the upper surface of an airfoil, so that the fabric assumes such contour in response to air blowing against the fabric. Optionally, a fan may be provided, desirably to blow air upwardly along the fabric.

In still yet another one of its many aspects, as illustrated generally in FIG. 13, the invention provides passive apparatus for improving indoor air quality where the apparatus includes a frame comprising vertically upstanding members positioned at the corners of an imaginary rectangle, one edge of the rectangle being considered the front of the rectangle. One pair of members has a first member of the pair at the right front of an associated rectangle and a second member of the pair at the right rear of the associated rectangle, with a second pair of members having a first member of the pair at the left front of the associated rectangle and a second member of the pair at the left rear of the associated rectangle. First and second horizontally oriented support members are preferably positionable on a floor or other surface to provide vertical support for the apparatus, and preferably respectively extend transversely between the lower extremities of the first and second members of the respective pairs of upstanding members. A plurality of vertically spaced apart pairs of parallel bracing members preferably connect respective ones of the upstanding first and second members of the respective pairs of upstanding members along respective sides of the rectangle, extending transversely away from the front. An uppermost lateral member extends between and preferably slideably engages uppermost correspondingly positioned pairs of the horizontally extending parallel bracing members. A plurality of lower lateral members below the uppermost lateral member, extend between and preferably slideably engage vertically correspondingly positioned pairs of the horizontally extending parallel bracing members. Air permeable, preferably naturally occurring botanically based antimicrobial, morbidity-inducing fabric is connected to the uppermost one of the lateral members and extends downwardly along and preferably between vertically adjacent pairs of lower lateral members. The lower lateral members are preferably movably positionable along the parallel bracing members, between front and rear, to cause the fabric portions connected thereto and extending therebetween to conform to selected contours. The lateral members may be positioned so that a portion of the resulting contour, or all of the resulting contour, approximates the upper surface of an airfoil, with the fabric conforming thereagainst in response to air blowing against the fabric. Similarly to the embodiment illustrated in FIG. 12, optimally a fan may be provided to blow air along or against the fabric.

In still yet another one of its many aspects, as illustrated in generally in FIG. 14, the invention provides passive apparatus for improving indoor air quality where the apparatus includes a frame comprising preferably vertically upstanding members preferably positioned at the corners of an imaginary rectangle, one edge of the rectangle being considered the front. One pair of members has a first member of the pair at the right front of the rectangle and the second member of the pair at the right rear of the rectangle, and a second pair of members has a first member of the pair at the left front of the rectangle and a second member of the pair at the left rear of the rectangle. Preferably, first and second horizontally oriented support members are preferably positionable on a floor or other surface to provide vertical support for the apparatus and respectively extend transversely between lower extremities of the first and second members of the respective pairs of upstanding members. A plurality of vertically spaced apart pairs of parallel bracing members preferably connect respective ones of the upstanding first and second members of the respective pairs of upstanding members along respective sides of the rectangle. Optionally, lateral members extend between and slideably engage vertically correspondingly positioned pairs of the horizontally extending parallel bracing members. Air permeable, preferably naturally occurring botanically based antimicrobial, morbidity-inducing fabric is connected to and extends between vertically adjacent pairs of bracing members. The optional lateral members are movably positionable along the parallel bracing members, between front and rear, to cause fabric portions to conform to selected contours, a portion of or all of which may approximate the upper surface of an airfoil, in response to air blowing against the fabric. Similarly to the embodiments illustrated in FIGS. 12 and 13, optionally a fan may be provided to blow air along or against the fabric.

### BRIEF DESCRIPTION OF THE DRAWINGS

FIG. 1 is an exploded isometric drawing of a first preferred embodiment of a modular unit for improving indoor air quality in accordance with aspects of the invention.

FIG. 2 is an exploded isometric drawing of a second preferred embodiment of a modular unit for improving air quality in accordance with aspects of the invention.

FIG. 3 is an exploded isometric drawing of a third preferred embodiment of a modular unit for improving indoor air quality in accordance with aspects of the invention.

FIG. 4 is an exploded isometric drawing of a fourth preferred embodiment of a modular unit for improving indoor air quality in accordance with aspects of the invention.

FIGS. 5 and 6 are exploded isometric drawings of preferred fifth and sixth preferred embodiments of modular units for improving indoor air quality in accordance with aspects of the invention.

FIG. 7 is an exploded isometric drawing of a seventh preferred embodiment of a modular unit for improving indoor air quality in form of a breathing light shelf, in accordance aspects of the invention.

FIG. 8 is an exploded isometric drawing of an eighth preferred embodiment of a modular unit for improving indoor air quality in the form of a breathing light shelf, similar to that shown in FIG. 7, in accordance with aspects of the invention.

FIG. 9 is an exploded isometric drawing of a ninth preferred embodiment of a modular unit for improving indoor air quality in the form of a breathing light shelf, similar to the embodiments illustrated in FIGS. 7 and 8, in accordance with aspects of the invention.

FIG. 10 is an exploded isometric drawing of a modular unit for improving indoor air quality in the form of a suspended breathing light shelf in accordance with aspects of the invention.

FIG. 11 is an exploded isometric drawing of a modular unit for improving indoor air quality in the form of a suspended breathing light shelf, similar to that illustrated in FIG. 10, in accordance with aspects of the invention.

FIG. 12 is an isometric drawing of a twelfth preferred embodiment of apparatus for preferably passively improving indoor air quality in the form of an upstanding modular vertically suspended fabric array, in accordance with aspects of the invention.

FIG. 13 is an isometric drawing of a thirteenth preferred embodiment of apparatus for preferably passively improving indoor air quality in the form of an upstanding modular vertically suspended fabric array, similar to that illustrated in FIG. 12, in accordance with aspects of the invention.

FIG. 14 is an isometric drawing of a fourteenth preferred embodiment of apparatus for preferably passively improving indoor air quality in the form of an upstanding modular vertically suspended fabric array, similar to that illustrated in FIGS. 12 and 13.

FIG. 15 is a broken isometric drawing of one of the five vertically extending segments of the apparatus for preferably passively improving indoor air quality illustrated in FIG. 12.

FIG. 16 is a broken isometric drawing of one of the five vertically extending segments of the apparatus for preferably passively improving indoor air quality illustrated in FIG. 14.

### DETAILED DESCRIPTION OF THE PREFERRED EMBODIMENTS OF THE INVENTION AND THE BEST MODE KNOWN AT THE PRESENT TIME FOR THE PRACTICE OF THE SAME

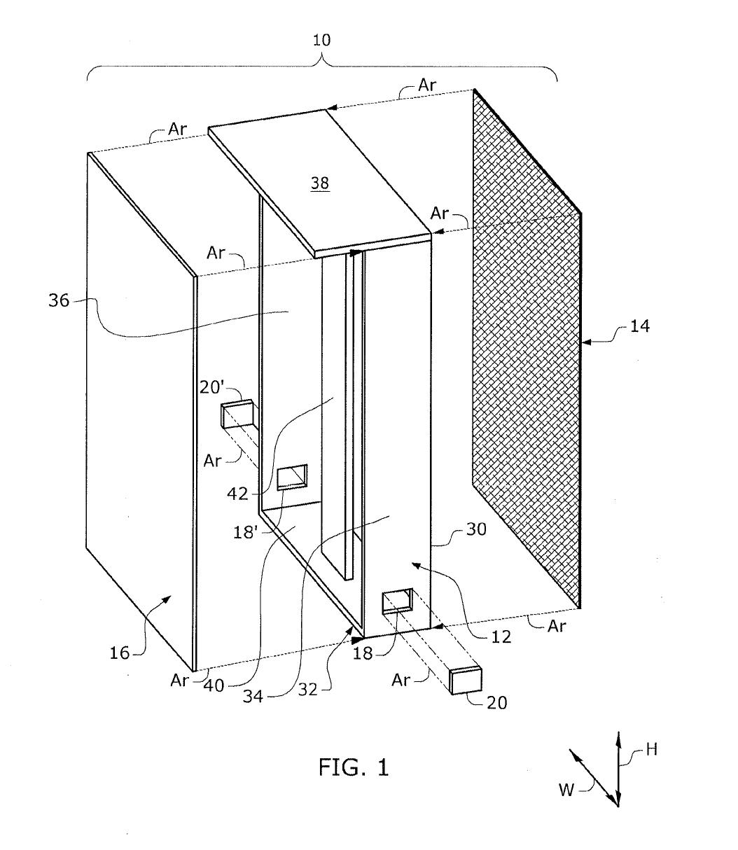

Referring generally to the drawings and specifically to FIG. 1, apparatus for treating and alleviating SBS to improve indoor air quality is depicted in the form of a modular unit designated generally 10 that includes a frame designated generally 12 surrounding an open interior and defining an outer periphery of unit 10. An air permeable, antimicrobial, morbidity-inducing fabric 14, preferably comprising a naturally occurring antimicrobial botanical compound, is secured about the periphery of frame 12, on a first side 30 of frame 12, with fabric 14 covering the open interior of frame 12 on the first side 30 of frame 12.

An air impermeable member designated generally 16 is secured about the periphery of frame 12 on a remaining or second side 32 of frame 12. Air impermeable member 16 covers the periphery of frame 12 on second side 32 of frame 12 completely about the frame periphery.

At least one aperture 18 is formed in frame 12. Aperture 18 is adapted to house a fan 20 therein. Fan 20 is depicted schematically in FIG. 1 and is preferably battery-powered and self-controlling. A second aperture 18′ may also be provided, as illustrated, to house a second optional fan 20′ or may be at least partially open and used for air bleed.

Fan 20, being housed in aperture 18, serves to blow air from outside of frame 12 into the interior of frame 12 for subsequent passage of substantially all air blown into the frame interior, outwardly through fabric 14.

In viewing FIG. 1, arrows identified by letters “Ar” indicate the manner of assembly of unit 10, which is shown in FIG. 1 in a partially exploded isometric view.

Still referring to FIG. 1, frame 12 has four members, two which are first and second upstanding lateral members 34 and 36, which are spaced apart as illustrated in FIG. 1; the remaining two members of frame 12 are a top member 38 and a bottom member 40.

Frame 12 further includes a bracing member 42 that is preferably upstanding and extends between and is affixed both to bottom member 40 and to top member 38 of frame 12. Bracing member 42 is preferably affixed to both top member 38 and bottom member 40 by screws, or by suitable adhesive, or by other mechanical fastening means. Similarly, frame 12 of unit 10 is preferably assembled from particle board or wood using adhesive or screws or other mechanical means to secure the parts of frame 12 together in the manner indicated by arrows Ar in FIG. 1. The screws, adhesive or other mechanical means used in the assembly of frame 12 have not been illustrated in FIG. 1, to enhance clarity of the drawing. As seen in FIG. 1, frame 12 is preferably of generally rectangular configuration, with frame 12 preferably being higher than it is wide.

The air permeable, antimicrobial, morbidity-inducing fabric 14, preferably comprising a naturally occurring antimicrobial botanical compound, is preferably secured about the edges of frame 12 that face fabric 14 when fabric 14 and frame 12 are oriented in the position illustrated in FIG. 1. Velcro is preferably used to secure fabric 14 to the surfaces of frame 12 that face fabric 14 when those parts are oriented as illustrated in FIG. 1. The Velcro has not been illustrated to enhance clarity of the drawing. Use of Velcro facilitates replacement of fabric 14 on a periodic basis.

When unit 10 is assembled by putting the parts of frame 12 in place as indicated by arrows Ar, including by positioning motor 20 within aperture 18, and by attaching fabric 14 to the facing edges of frame 12 using the preferable Velcro, other than for the presence of upstanding bracing member 42, the interior of frame 12 is open.

Upstanding bracing member 42 is of width substantially less than the width of the top and bottom 38 and 40 of frame 12, where width is considered in the direction indicated by arrow W in FIG. 1 and height is considered in the direction indicated by arrow H in FIG. 1. With upstanding bracing member 42 being of width substantially less than top and bottom 38 and 40 of frame 12, when frame 12 is assembled, the interior of frame 12 is essentially open, thereby providing a plenum that is at least partially bounded by fabric 14. When fan 20 operates, fan 20 introduces air into the plenum defined by the interior of unit 10 and forces air gently outwardly through fabric 14. Fabric 14 is air permeable and has antimicrobial, morbidity-inducing characteristics due to having been treated with and preferably even impregnated with a naturally occurring antimicrobial botanical material, preferably clove powder or eugenol. Hence, when room air is forced gently into the open interior of unit 10, which defines a plenum, and then outwardly through fabric 14, airborne bacteria and other contaminants are trapped and killed by fabric 14.

As further apparent from FIG. 1, frame 12 has a generally rectangular configuration such that first side 30 and second side 32 are parallel one with another, and such that top 38 and bottom 40 are parallel one with another. Additionally, the edges, which are unnumbered in the drawings, of first and second sides 30, 32 and top and bottom 38, 40, are all coplanar, thereby presenting a flat, rectangular, frame-like surface for preferable adhesive affixment of the Velcro male or female portion that mates with the counterpart Velcro portion affixed to fabric 14. Fabric 14 is preferably of rectangular shape and dimensioned to fit congruently with the facing edges of first and second sides 30, 32 and top and bottom 38, 40, defining the rectangular shape of frame 12, so that there is no substantial overlap of fabric 14 respecting frame 12, and so that there is no opening between an edge of fabric 14 and a portion of frame 12 through which air could escape without passing through fabric 14.

From the foregoing description of the structure illustrated in FIG. 1, together with the image provided by FIG. 1, it is apparent that unit 10 may be used to combat SBS and includes a plenum defined by the interior of frame 12, which is at least partially bounded by fabric 14 where fabric 14 has at least one preferably naturally occurring botanical substance exhibiting antimicrobial, microorganism morbidity-inducing properties, and where unit 10 further includes a fan for introducing air into the plenum for passage of the air outwardly through the fabric, thereby to trap and to kill airborne bacteria and other contaminants contributing to SBS. It is further apparent that operation of unit 10 inherently practices a method for combating SBS by providing a plenum, namely the interior of frame 12, which is at least partially bounded by fabric, namely the air permeable, antimicrobial, morbidity-inducing fabric 14, preferably comprising a naturally occurring antimicrobial botanical compound, where the method further includes introducing air into the plenum for passage outwardly through the fabric. It is still further apparent that operation of unit 10 inherently practices a method for combating SBS by forcing air from a room contaminated by SBS through a fabric where the fabric has at least one preferably naturally occurring botanical substance exhibiting microorganism morbidity-inducing properties.

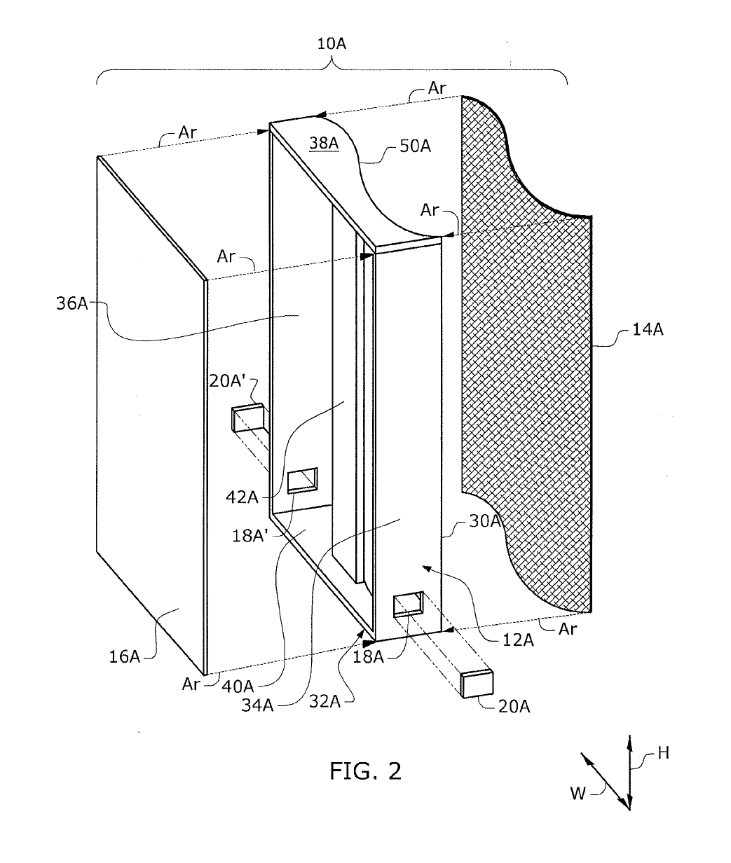

Still referring generally to the drawings and specifically to FIG. 2, apparatus for treating and eliminating SBS and improving indoor air quality is depicted in the form of a modular unit designated generally 10A. Unit 10A is similar to unit 10 illustrated in FIG. 1 with the letter “A” used in FIG. 2 to identify the apparatus illustrated in FIG. 2. Components illustrated in FIG. 2 having the same function or effectively identical to components in FIG. 1 have the same numbers in FIG. 2 as in FIG. 1.

The apparatus illustrated in FIG. 2 differs from the apparatus illustrated in FIG. 1 in that the top 38A of frame 12A and the bottom 40A of frame 12A, have edges that are not straight as illustrated in FIG. 1 but are curved in somewhat the shape of an “S” curve, as the outwardly facing edge of top 38A is clearly visible in FIG. 2. This forwardly or outwardly facing edge of top 38A is designated 50A in FIG. 2. Providing forwardly facing edge 50A in a curved, desirable S-shaped configuration permits use of air permeable, antimicrobial, morbidity-inducing fabric 14A, preferably comprising a naturally occurring antimicrobial botanical compound, having greater surface area in the embodiment illustrated in FIG. 2 than in the embodiment illustrated in FIG. 1, with the footprint of unit 10A, namely the floor space occupied by unit 10A, remaining the same as the footprint of unit 10 illustrated in FIG. 1. As a result, unit 10A in FIG. 2 may have greater air treatment capacity for a given amount of floor space occupied by unit 10A than the air treatment capacity of unit 10, as illustrated in FIGS. 2 and 1, respectively. Additionally, the forwardly facing edge 50A with the curved, S-shaped configuration provides even greater aesthetics for unit 10A illustrated in FIG. 2 relative to unit 10 illustrated in FIG. 1.

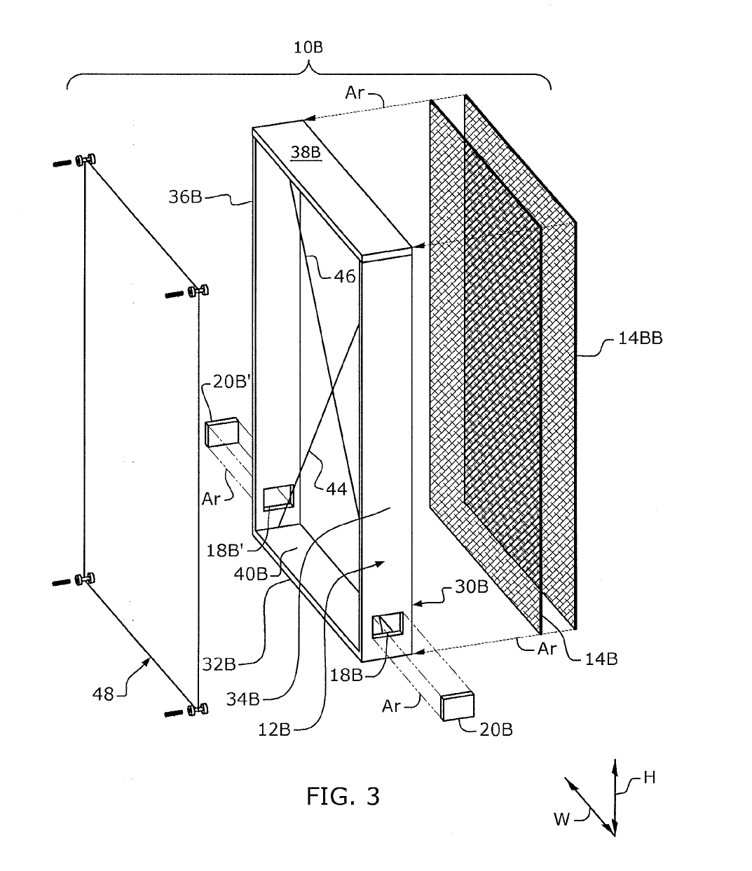

Still referring generally to the drawings and specifically to FIG. 3, another embodiment of apparatus for treating and alleviating SBS thereby improving indoor air quality is depicted in the form of a modular unit designated generally 10B that includes a frame designated generally 12B surrounding an open interior and defining an outer periphery of unit 10B. In FIG. 3, the same lettering and numbering convention as used in FIGS. 1 and 2 has been employed. Specifically, components having the same or substantially the same function and generally corresponding substantially to components illustrated in FIGS. 1 and 2 have the same number; the letter “B”, or in some cases the letters “BB”, is used to denote FIG. 3 and the embodiment therein and to distinguish the same from the embodiments illustrated in FIGS. 1 and 2. In FIG. 3, two layers 14B and 14BB of air permeable, antimicrobial, morbidity-inducing fabric, preferably comprising a naturally occurring antimicrobial botanical compound are secured about the periphery of frame 12B on a first side 30B of frame 12B with fabric 14B facingly contacting the open interior of frame 12B on first side 30B of frame 12B, and with fabric 14BB facingly contacting fabric 14B and lying congruently thereover.

Similarly to the apparatus illustrated in FIGS. 1 and 2, at least one aperture 18B is formed in frame 12B. Aperture 18B is adapted to house a fan 20B therein. Fan 20B is depicted schematically in FIG. 3. A second aperture 18B′ may also be provided as illustrated to house a second optional fan 20B′ or may be used for air bleed.

Fan 20B, being housed in aperture 18B, serves to blow air from outside of frame 12B into the interior of frame 12B for subsequent passage of substantially all air that is blown into the frame interior, outwardly through fabric 14B and 14BB.

As with FIGS. 1 and 2, the arrows identified in FIG. 3 by letters “Ar” indicate the manner of assembly of unit 10B, which is shown in FIG. 3 in a partially exploded isometric view.

The remaining or second side 32B of frame 12B may be open as illustrated, or may be covered with one or more layers of air permeable, antimicrobial, morbidity-inducing fabric.

Still referring to FIG. 3, frame 12B has four members, two of which are first and second upstanding lateral members 34B and 36B, which are spaced apart as illustrated in FIG. 3; the remaining two members of frame 12B are top member 38B and bottom member 40B.

Frame 12B further preferably includes first and second diagonal bracing cables 44 and 46, each of which extend from a lower interior corner of frame 12B, defined by juncture of bottom 40B and upstanding side member 34B or 36B, to a diagonally opposite upper corner, defined by juncture of top 38B with either upstanding side member 36B or upstanding side member 34B. Diagonal bracing cables 44 and 46 are secured in place, desirably by connecting with eyes driven into the wood or particle board construction, at a location close to if not exactly at the line of juncture between the top and bottom members 22B, 24B and the respective side members 34B, 36B. The eyes and the particular securement of diagonal bracing cables 44 and 46 to frame 12B have not been illustrated in FIG. 3 to enhance drawing clarity.

Unlike the unit illustrated in FIG. 1, the remaining or second side 32B of frame 12B in the unit illustrated in FIG. 3 has been illustrated open. Unit 10B is preferably equipped with a hanging cable 48 connected to second side 32B of frame 12B by suitable screw and collar assemblies, which have not been detailed or numbered in FIG. 3 to enhance drawing clarity. As shown in FIG. 3, screws are driven into the second side 32B of frame 12B at the four corners of second side 32B and collars are then secured in place by screws and permit a small degree of movement of hanging cable 48. Presence of hanging cable 48 facilitates hanging unit 10B on and against a wall, with the wall thereby effectively closing second side 32B of frame 12B in much the same manner as air permeable member 16 covers the periphery of frame 12 on the second side 32 of frame 12 as illustrated in FIG. 1.

Hanging cable 48 and the unnumbered screws and collars that connect hanging cable 48 to the remainder of the structure may also and optionally be positioned to maintain the frame 12B slightly away from the wall on which unit 10B is mounted. This is desirable if the remaining or second side 32B of frame 12B is covered with one or more layers of air permeable, antimicrobial, morbidity-inducing fabric, preferably comprising a naturally occurring antimicrobial botanical compound. Unit 10B, using hanging cable 48, can be mounted against any reasonably imperforate wall surface; provision of hanging cable 48 permits unit 10B to be mounted essentially flush against the surface of the wall on which unit 10B is mounted. Molly bolts, hooks or the like, driven into a wall may be used to hang unit 10B on the wall.

While unit 10B has been illustrated with two thicknesses of air permeable, antimicrobial, morbidity-inducing fabric 14B and 14BB, a single fabric thickness may be used, depending on the amount of air moved by fan 20B as selected in specifying fan 20B. While one or more layers of air permeable, antimicrobial, morbidity-inducing fabric, preferably comprising a naturally occurring antimicrobial botanical compound, 14B, 14BB may be used on the front and rear surfaces of frame 12B, an aesthetically pleasing, air permeable fabric lacking antimicrobial and morbidity-inducing properties may be used as the outermost fabric 14BB to enhance the aesthetics of unit 10B.

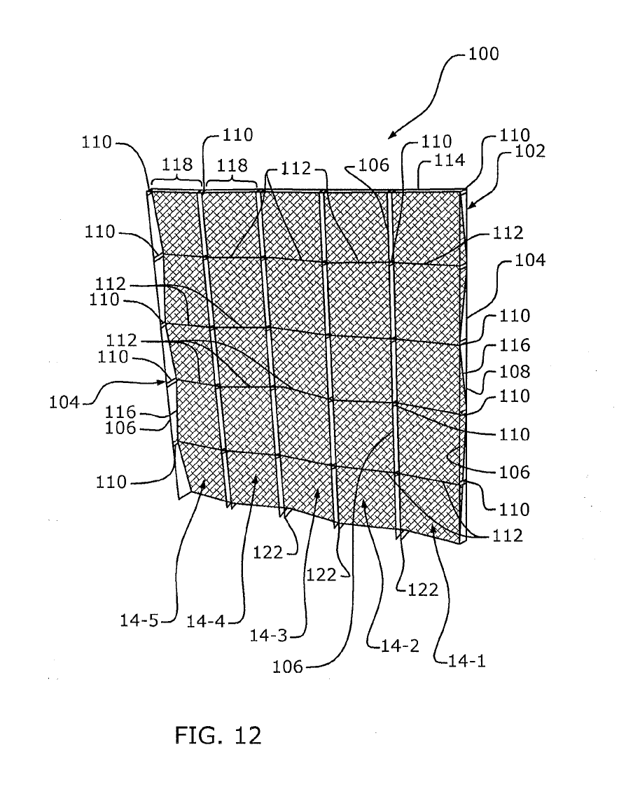

Similarly to the unit illustrated in FIG. 1, frame 12B of unit 10B is preferably assembled from particle board or wood using adhesive, screws or other mechanical means to secure the parts of frame 12B together in the manner indicated by arrows Ar in FIG. 3. As in FIG. 1, the screws, adhesive or other mechanical means used in the assembly of frame 12B have not been illustrated in FIG. 3 to enhance clarity of the drawing. Similarity to the apparatus illustrated in FIGS. 1 and 12, frame 12B is preferably of generally rectangular configuration with frame 12 preferably being higher than it is wide.

The air permeable, antimicrobial, morbidity-inducing fabric 14B is preferably secured about the edges of frame 12B that face fabric 14B when fabric 14B and frame 12B are oriented in the position illustrated in FIG. 3. As with the apparatus illustrated in FIGS. 1 and 2, Velcro is preferably used to secure fabric 14B to the surfaces of frame 12B that face fabric 14B when those parts are oriented as illustrated in FIG. 3. Similarly, Velcro is preferably used to secure fabric 14BB to the surface of fabric 14B when those fabric layers are oriented as illustrated in FIG. 3. The Velcro has not been illustrated in order to enhance the drawing. Use of Velcro facilitates replacement of the fabrics on a periodic basis.

Similarly to the apparatus illustrated in FIGS. 1 and 2, when unit 10B is assembled by putting the parts of frame 12B in place as indicated by arrows Ar, by positioning motor 20B with an aperture 18B, and by attaching fabric 14B and 14BB to the facing edges of frame 12B using the preferable Velcro, and unit 10B is either mounted flushly against a wall or has fabric 14BBB covering the rear or second side of unit 10B, the interior of frame 12B is open other than for the presence of diagonal bracing cables 44, 46. The open construction provides a plenum that is at least partially bounded by fabric 14B. When fan 20 operates, fan 20 introduces air into the plenum defined by the interior of unit 10B and forces air gently outwardly through fabric 14B and fabric 14BB. Fabrics 14B and 14BB are both air permeable and preferably each has antimicrobial, morbidity-inducing characteristics due to having been treated with, and even impregnated with, a naturally occurring botanical, antimicrobial, morbidity-inducing material, preferably clove powder or eugenol. Hence, when room air is forced gently into the open interior of unit 10B, which defines a plenum, and then outwardly through fabric 14B, 14BB, airborne bacteria and other contaminants are trapped and killed by fabric 14B and 14BB.

As also apparent from FIG. 3, frame 12B has a generally rectangular configuration such that first side 30B and second side 32B are parallel one with another and such that top 38B and bottom 40B are parallel one with another. Additionally, the edges, which are unnumbered in the drawings, of the first and second sides 30B, 32B and top and bottom 38B, 40B are all coplanar, thereby presenting a flat, rectangular, frame-like surface for preferable adhesive securement of the Velcro male or female portion that mates with the counterpart Velcro portion affixed to fabric 14B. Fabric 14B and fabric 14BB are both preferably rectangularly shaped and dimensional to fit congruently with the facing edges of first and second sides 30B, 32B and the facing edges of top and bottom 38B, 40B defining the rectangular shape of frame 12B so there is no substantial overlap of fabric 14B, 14BB, respecting frame 12, and so there is no opening between an edge of fabric 14B and a portion of frame 12B through which air could escape without passing through fabric 14B.

Similarly to the apparatus illustrated in FIGS. 1 and 2, from the foregoing description in combination with the image provided by FIG. 3, it is apparent that unit 10B may be used to combat SBS and includes a plenum defined by the interior of frame 12B when frame 12B is closely spaced and perhaps even contacting the wall on which frame 12B is mounted, with the plenum at least being partially bounded by fabric 14B, where fabric 14B has at least one preferably naturally occurring botanical substance exhibiting antimicrobial, microorganism morbidity-inducing properties, and where unit 10B further includes a fan for introducing air into the plenum for passage of the air outwardly through the fabric, thereby to trap and kill airborne bacteria and other contaminants contributing to SBS. It is further apparent that operation of unit 10B inherently practices a method for combating SBS by providing a plenum, namely the interior of frame 12B, which is at least partially bounded by fabric, namely the air permeable, antimicrobial, morbidity-inducing fabric 14B, where the method further includes introducing air into the plenum for passage outwardly through the fabric.

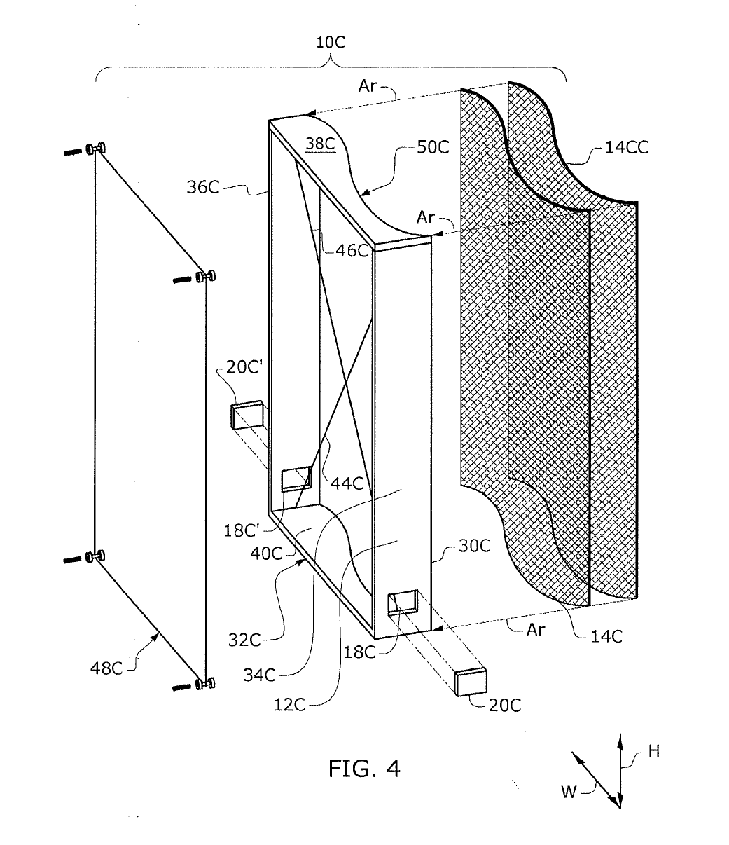

Still referring generally to the drawings and specifically to FIG. 4, yet another embodiment of apparatus for treating and alleviating SBS thereby improving indoor air quality is depicted in the form of a modular unit designated generally 10C that includes a frame designated generally 12C surrounding an open interior and defining an outer periphery of unit 10C. In FIG. 4, the same lettering and numbering convention has been used for the apparatus therein as used above for the apparatus illustrated in FIGS. 1, 2 and 3, in that elements or components of apparatus 10C that are identical to or functionally corresponding with elements or components in FIGS. 1, 2 and 3 have the same number, with letter “C” identifying and distinguishing the apparatus illustrated in FIG. 4 from the apparatus illustrated in FIGS. 1, 2 and 3.

In apparatus 10C illustrated in FIG. 4, much like apparatus 10A illustrated in FIG. 2, the forwardly facing edges of top 38C and bottom 40C of frame 12C are curved, preferably in an S-shape configuration, as illustrated by the forwardly facing edge 50C of top 38C in FIG. 4. This, similarly to the curved forwardly facing edge configuration in FIG. 2, facilitates use of a larger surface area of fabric 14C, 14CC, in a given floor area, where the footprint of unit 10C is the same as that of unit 10B. In all other respects, the descriptive material set forth above respecting FIG. 3 is incorporated herein by reference with respect to FIG. 4.

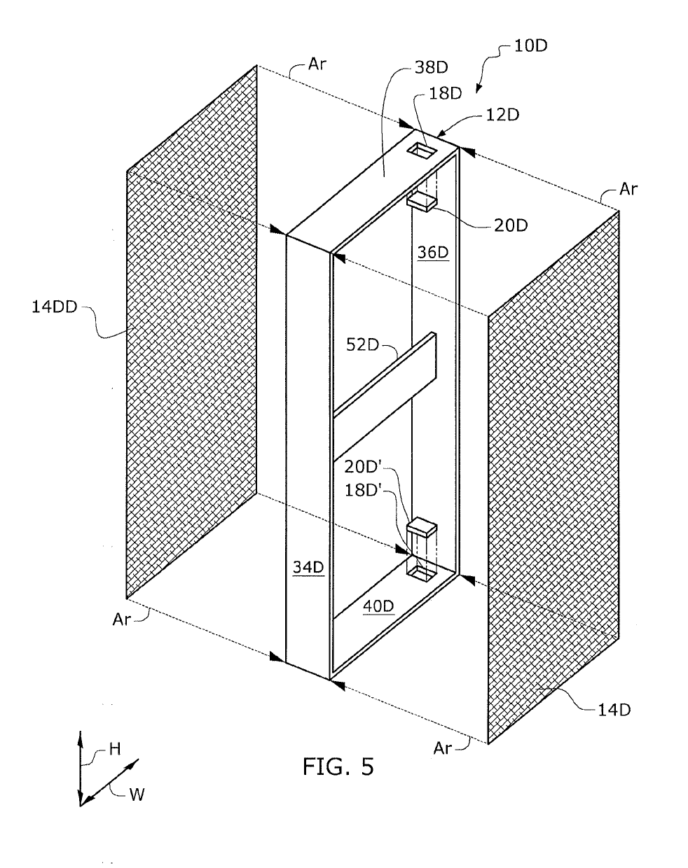

Still referring generally to the drawings and specifically to FIG. 5, yet another embodiment of apparatus for treating and alleviating SBS and thereby improving indoor air quality is depicted in the form of a modular unit designated generally 10D that includes a frame designated generally 12D surrounding an open interior and defining an outer periphery of unit 10D. The same numbering convention has been used in FIG. 5 as used in FIGS. 1 through 4 in that elements of apparatus 10D that are identical to or functionally correspond with elements in FIGS. 1 through 4 have the same numbers and the letter “D” is used to identify and distinguish parts and components of unit 10D as illustrated in FIG. 5 from parts and components illustrated in FIGS. 1 through 4.

Unit 10D illustrated in FIG. 5 differs from the apparatus illustrated in FIGS. 1 through 4 in that the apparatus illustrated in FIG. 5 is not intended to rest on the floor, but rather is intended to be hung in a freestanding manner within a room requiring remediation and cure of SBS symptoms. As illustrated in FIG. 5, bottom member 24D of frame 12D is equipped with an aperture 18D′ into which a fan 20D′ may be positioned. Similarly, top member 22D of frame 12D includes an aperture 18D into which a fan 20D may be positioned. Additionally, unit 22D preferably includes two air permeable, antimicrobial, morbidity-inducing fabrics, preferably comprising a naturally occurring antimicrobial botanical compound, 14D and 14DD, with one fabric fitting on each side of frame 12D. Arrows Ar identify the manner in which unit 10D is assembled with fabrics 14D, 14DD fitting against frame 12D. Not all of the arrows denoting such assembly have been labeled “Ar” in order to enhance drawing clarity. Frame 12D further includes horizontal interior bracing member 52D to provide rigidity to frame 12D when assembled. With the exception of horizontal interior bracing member 52D, the interior of frame 12D is open when unit 12D is assembled.

As respecting operation of unit 10D, the information and description as provided above as respecting units 10, 10A, 10B and 10C illustrated in FIGS. 1 through 4 is hereby incorporated by reference as being equally applicable to unit 10D illustrated in FIG. 5.

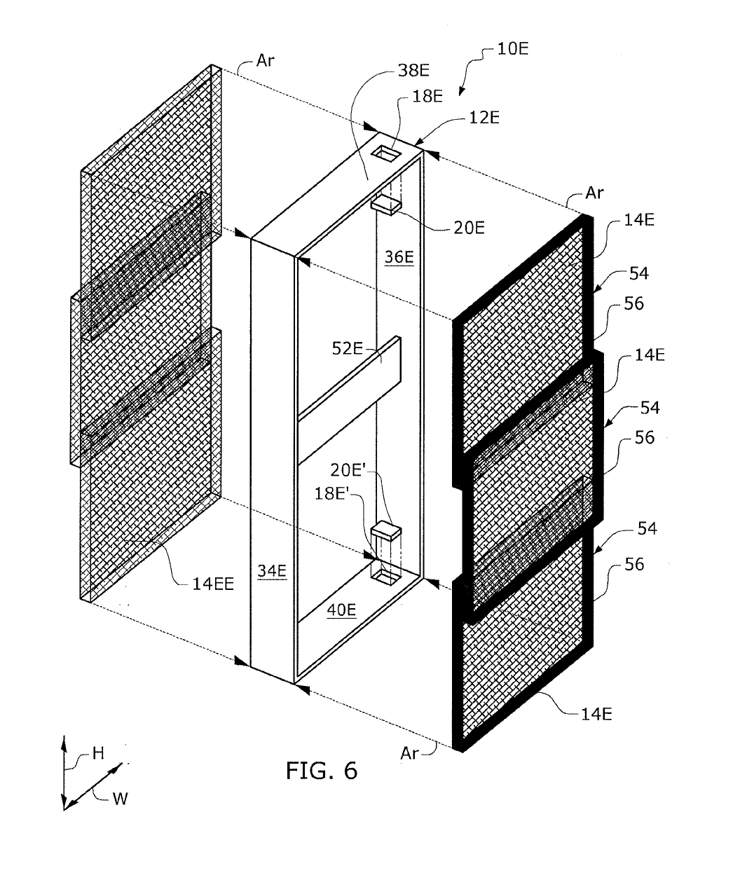

Still referring generally to the drawings and specifically to FIG. 6, yet another embodiment of apparatus for treating and alleviating SBS thereby improving indoor air quality is depicted in the form of a modular unit designated generally 10E that includes a frame designated generally 12E surrounding a generally open interior and defining an outer periphery of unit 10E. In FIG. 6, the same numbering and lettering convention as used with respect to the apparatus illustrated in FIGS. 1 through 5 has been adopted; in FIG. 6, the letter “E” identifies and distinguishes components of unit 10E illustrated therefrom from the corresponding parts and components of the apparatus illustrated in FIGS. 1 through 5. Referring still to FIG. 6, frame 12E and the parts thereof, namely top member 22E, bottom member 24E, lateral members 26E, horizontal interior bracing member 52E, fans 20E and 20E′ and apertures 18E and 18E′ are preferably substantially identical to the correspondingly numbered components of unit 10D illustrated in FIG. 5.

In FIG. 6, the air permeable, antimicrobial, morbidity-inducing fabric is furnished in the form of modular fabric panels designated generally 54 in FIG. 6, where each modular fabric panel includes a frame 56 that is generally of rectangular construction with an open center. Preferably two layers of air permeable, botanically based, antimicrobial, morbidity-inducing fabric 14E and 14EE are a part of each modular fabric panel 54 with a first layer of fabric 14E secured to one side of frame 56 and a second layer of fabric 14EE secured to a second side of frame 56, where the fabric in both instances is preferably secured to frame 56 using Velcro. In FIG. 6, to enhance drawing clarity, the frames 56 of modular fabric panels 54 have been illustrated only for modular fabric panels 54 on the right side of the drawing. Similarly, fabric layer 14E has been designated only for those modular fabric panels on the right side of the drawing and fabric layer 14EE has been designated only for those modular fabric panels on the left side of the drawing.

Each modular fabric panel preferably includes two layers of fabric, one on either side of fabric panel frame 56. Modular fabric panels 54 may be dimensioned such that when mounted on frame 12E there is some overlap of the upper and lower panels by the middle panel as illustrated in FIG. 6; unit 10E may also be constructed such that modular fabric panels 54 all collectively fit flushly one against another on one side of frame 12E to present a smooth, continuous surface of air permeable, antimicrobial, morbidity-inducing fabric, preferably comprising a naturally occurring antimicrobial botanical compound, for passage of treatment air therethrough.

As respecting the operation of unit 10E and the components thereof, the information and disclosure as set forth above with respect to the apparatus illustrated in FIGS. 1 through 5 is incorporated by reference.

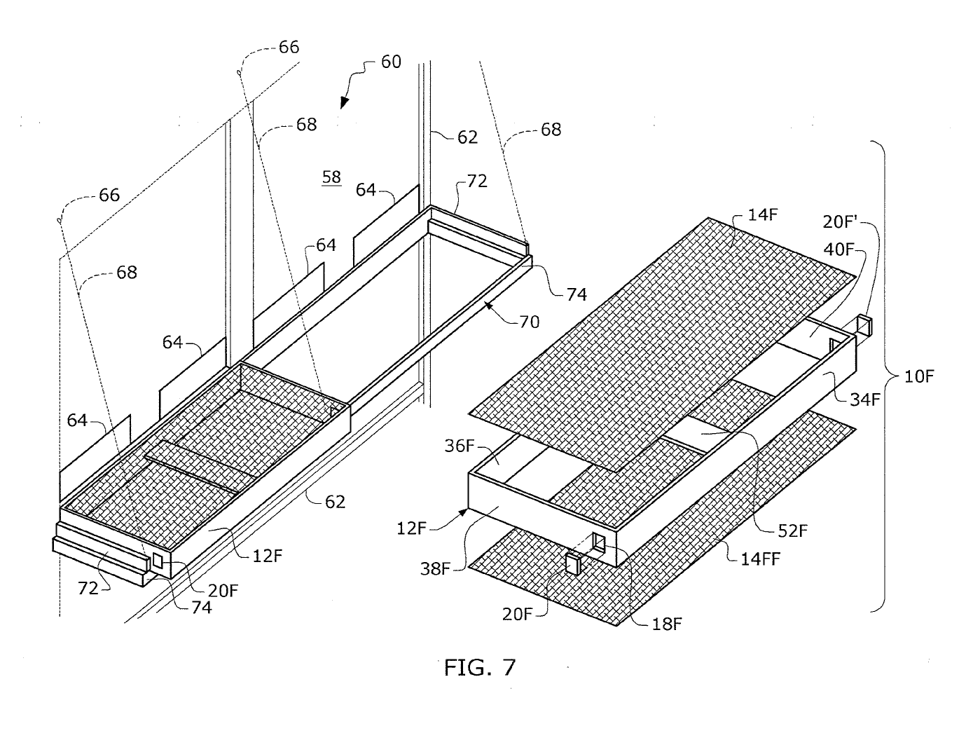

Still referring generally to the drawings and specifically to FIG. 7, yet another embodiment of apparatus for treating and alleviating SBS and improving indoor air quality is depicted in the form of a modular unit designated generally 10F that includes a frame designated generally 12F surrounding an open interior and defining an outer periphery of unit 10F. As with the apparatus illustrated in FIGS. 1 through 6, the same numbering and lettering convention has been used with the letter “F” being used to identify and distinguish components of the apparatus illustrated in FIG. 7 from corresponding functionally and substantially equivalent components of the apparatus illustrated in FIGS. 1 through 6. As illustrated in the right-hand, exploded view portion of FIG. 7, the apparatus for treating and alleviating SBS and improving indoor air quality appearing there and indicated as 10F is substantially identical to that illustrated in FIG. 5 and designated 10D. In FIG. 7, apparatus 10F has been illustrated in a horizontal disposition and, as shown in the left hand portion of FIG. 7, is adapted to be used in such a horizontal orientation.

As further illustrated in the left-hand portion of FIG. 7, unit 10F is preferably mounted in a horizontal disposition on a unit support frame designated generally 70 that is positioned within a structure designated generally 60 and preferably is in essentially facing contact with the interior surface of a window, or at least the frame of the window, designated generally 58. Unit support frame 70 is preferably maintained in place and vertically supported by cable 68 preferably connected to hooks 66 mounted in the interiorly facing surface of wall 62, above window 58.

Unit support frame 70 preferably includes an inner member designated generally 72 and an outer member designated generally 74 as shown in the left-hand portion of FIG. 7. Outer member 74 is dimensioned to vertically support unit 10F by contact with a downwardly facing portion thereof, preferably the downwardly facing portion of frame 12F of unit 10F, as illustrated at the extreme left-hand side of FIG. 7. Inner member 72 of unit support frame 70 is dimensioned to receive unit 10F in a facing, complemental manner with unnumbered vertically extending, horizontally facing surfaces of inner member 72 facingly contacting the interiorly positioned one of lateral members 26F and members 22F and 24F. The portion of inner member 72 extending essentially perpendicularly inwardly from window 58 is dimensioned to stop short of the position of fan 20F in aperture 18F, all as illustrated in the extreme left-hand portion of FIG. 7.

Solar cells 64 are preferably positioned in facing contact with window 58 to receive sunlight and thereby generate electricity. Solar cells 64 are connected by wires, not shown in the drawings, to fans 20F so that fans 20F are driven by solar energy received through window 58, such that batteries may not be required for fans 20F.

In one preferable implementation of the invention as illustrated in FIG. 7, fabric 14F on the upper side of unit 10F may be a non-woven fabric that is not only air permeable and antimicrobial with morbidity inducing properties, but is also reflective in a manner to reflect natural light coming in through window 58 throughout the room in which unit 10F is mounted. Distribution of natural light within a room having SBS symptoms serves to alleviate those symptoms and in combination with the air purification effectuated by unit 10F provides synergistic results as respecting elimination of SBS.

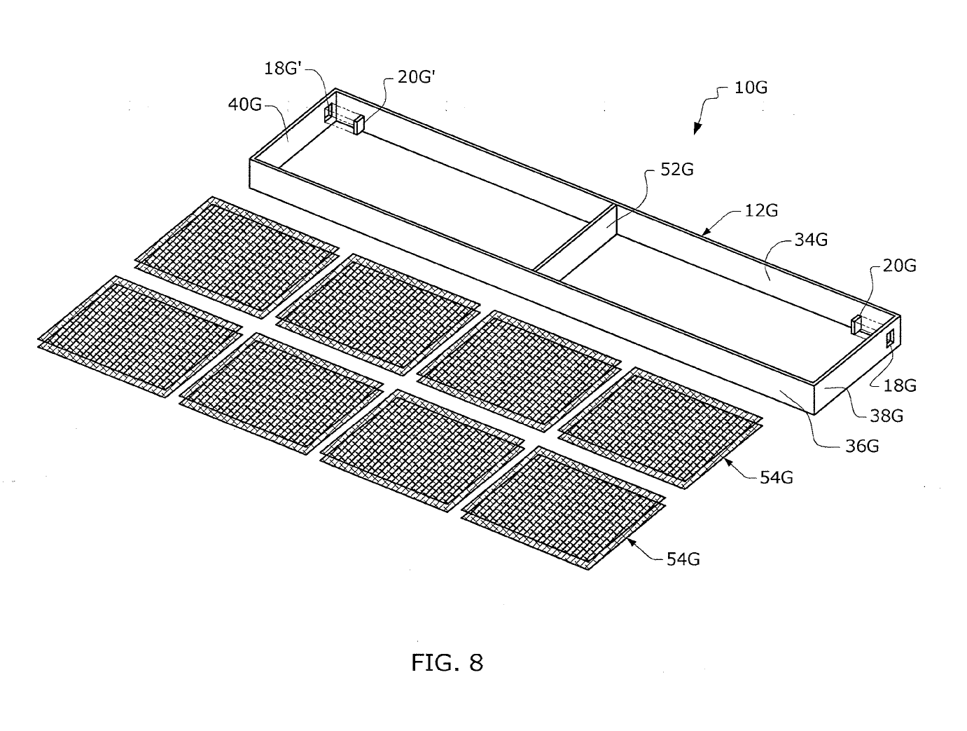

Still referring generally to the drawings and specifically to FIG. 8, apparatus for treating and alleviating SBS and improving indoor air quality is depicted in the form of a modular unit designated generally 10G that includes a frame designated generally 12G surrounding an open interior and defining an outer periphery of unit 10G. Unit 10G is designed to fit on a unit support frame 70 illustrated in FIG. 7. The frame 12G of unit 10G is essentially identical to frame 12F illustrated in FIG. 7 except for the position of interior bracing member 52G which has been positioned to be in the same plane with and transverse to lateral members 26G of frame 12G.

As further illustrated in FIG. 8, the air permeable antimicrobial, morbidity inducing fabric, preferably comprising a naturally occurring antimicrobial botanical compound, is provided in the form of modular fabric panels 54G, as shown in FIG. 6 as panels 54. Frames of the modular panels 54G have not been illustrated in FIG. 8 to enhance drawing clarity. The same numbering convention has been used with respect to unit 10F illustrated in FIG. 8 as for the apparatus illustrated in FIGS. 1 through 7 and the disclosure as set forth above for the apparatus illustrated in FIGS. 1 through 7 and the operation thereof is hereby incorporated as applicable to apparatus 10G illustrated in FIG. 8. Not all of modular fabric panels 54G have been so-designated in FIG. 8 to enhance drawing clarity.

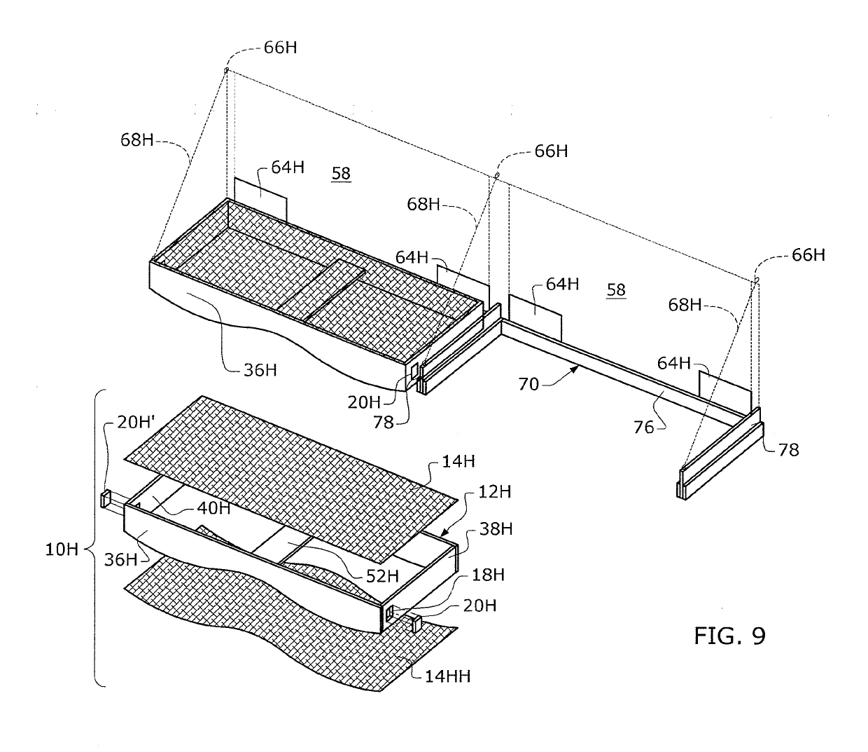

Still referring generally to the drawings and specifically to FIG. 9, apparatus for treating and alleviating SBS and improving indoor air quality is depicted in the form of a modular unit designated generally 10H that is similar to that illustrated in FIG. 7, with the exception that downwardly facing edges of lateral members 26 are formed in an S-curve in order to provide more surface area of air permeable, antimicrobial, morbidity-inducing fabric, preferably comprising a naturally occurring antimicrobial botanical compound, 14H, than the fabric surface area provided by the embodiment illustrated in FIG. 7 for a given area occupied by unit 10H. In all other respects apparatus illustrated in FIG. 9 as unit 10H is essentially identical to that illustrated in FIG. 7 as apparatus 10F, and the disclosure as set forth above respecting the apparatus of FIG. 7 is hereby substantially incorporated by reference with respect to the apparatus illustrated in FIG. 9. One exception to the incorporation by reference is a minor change to the configuration of unit support frame 70 in that outer member 74 illustrated in FIG. 7 does not extend across the front of unit support frame 70, as illustrated in FIG. 9. Rather, unit support frame 70 as illustrated in FIG. 9 includes a bottom support member 76 for supporting unit 10H by facing complemental contact with the lower surfaces of three sides of the frame 12H of unit 10H. Unit support frame 70 as illustrated in FIG. 9 includes a pair of lateral guide members 78 which facingly contact members 22H, 24H, as illustrated in the upper portion of FIG. 9, but are dimensioned so as to stop short of fan 20H, as illustrated in the upper portion of FIG. 9.

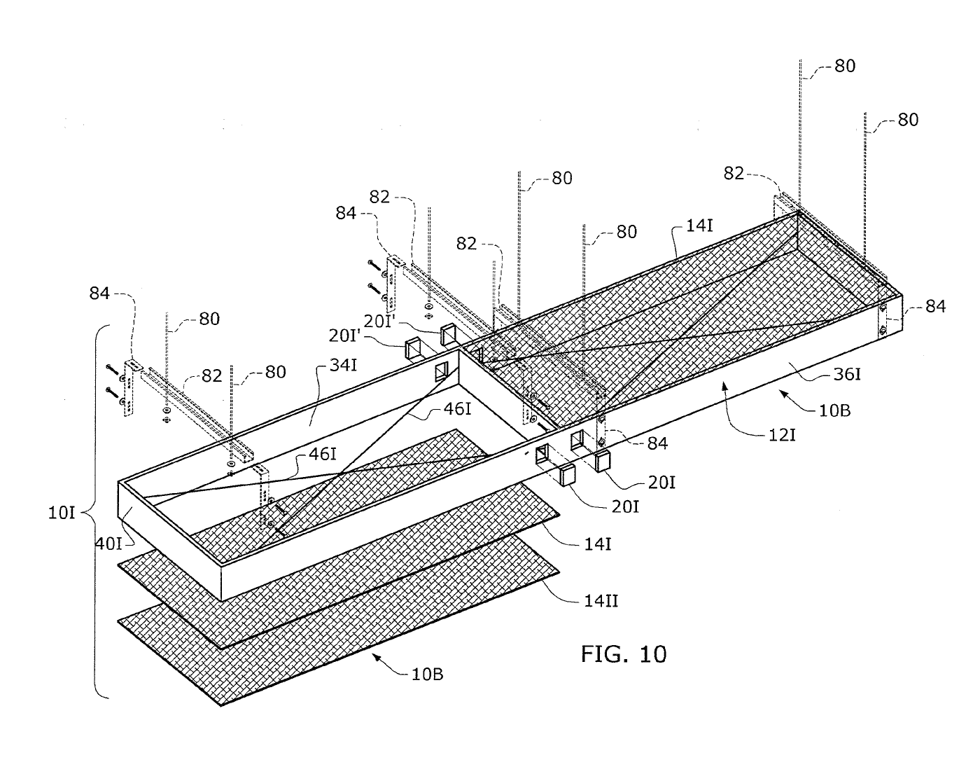

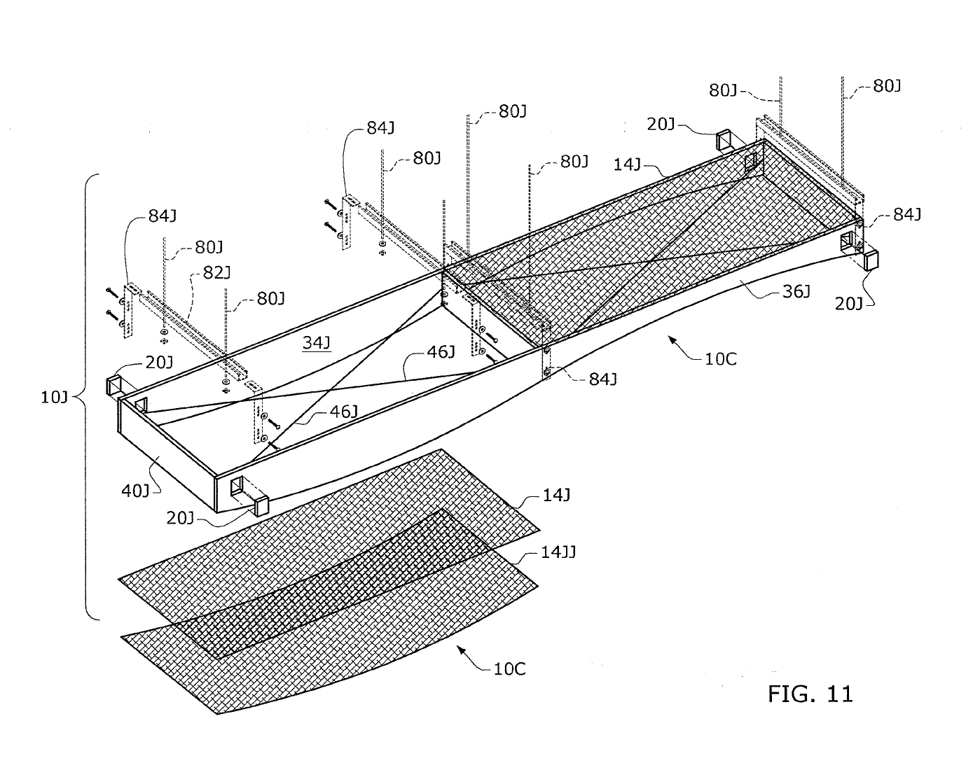

Continuing to refer to the drawings, FIGS. 10 and 11 illustrate use of the apparatus for treating and alleviating SBS thereby improving indoor air quality, as depicted in FIGS. 3 and 4 and identified as units 10B and 10C, in a horizontal orientation with the units being suspended in mid-air from the ceiling or other structure in a room in order to alleviate SBS. In FIG. 10, two units 10B are illustrated in side-by-side disposition, with one of the units, on the left side of FIG. 10, being shown with an open center. In use, the upper surface of unit 10B illustrated to the left in FIG. 10 would be covered, either with one or more layers of air permeable, antimicrobial, morbidity inducing fabric 14 or with a solid cover such as rear cover member 16 illustrated in FIG. 1.

FIG. 11 similarly shows two units 10C with the unit to the left-hand side of FIG. 11 having an open top which would be covered in the same manner as that of FIG. 10. In FIGS. 10 and 11, the composite units 10B, in the case of FIG. 10, or two units 10C in the case of FIG. 11, have been designated 10I and 10J respectively. In FIG. 10 vertically-elongated L-brackets are attached by nuts and bolts or by screws to lateral members 34I, 36I of frame 12I. The horizontal portions of L-brackets 84 fit into respective ends of C-channels 82. C-channels 82 are drilled to receive vertical struts 80 that are held in place by nuts and washers, not numbered but illustrated in FIG. 10. Vertical struts 80 are in turn suspended from the ceiling or other structure high in the room in which unit 10I is located and preferably maintain unit 10I at a level such that the lower surface of unit 10I is above the level of windows and/or eve skylights lighting fixtures supplying light to the room. In this regard it is preferable that the lowest sheet of fabric indicated as 14II in FIG. 10 be not only air permeable, antimicrobial and morbidity inducing, preferably comprising a naturally occurring antimicrobial botanical compound, but also be light reflective thereby to effectuate better distribution of light within the room in which unit 10I is installed. Light together with the air treatment provided by unit 10I provides synergistic effects with respect to elimination of SBS syndrome as noted above.

With respect to FIG. 11, unit 10G is essentially the same as unit 10I except for presence of the curved lower surface of members 26J. As noted above, such curvature provides greater surface area of air permeable, antimicrobial, morbidity inducing fabric for a given footprint, measured in square area units, of the unit 10J verses 10I. In other respects, unit 10J is the same as unit 10I and the disclosure as set forth above respecting unit 10I is hereby incorporated by reference for unit 10J as illustrated in FIG. 11.

Referring to FIG. 12, apparatus for preferably passively treating and alleviating SBS to improve indoor air quality is depicted in the form of a vertically upstanding array designated generally 100 that includes a frame designated generally 102 for supporting strips of air permeable, botanically based antimicrobial, morbidity-inducing fabric, where the strips of fabric are designated 14-1, 14-2, 14-3, 14-4 and 14-5. Frame 102 supporting fabric strips 14-1 through 14-5 includes a plurality of upstanding members that are individually designated generally 104. Upstanding members 104 are categorized as first and second upstanding members 106, 108 that are connected front to back by bracing members 110.

Extending laterally between pairs of bracing members 110 and being a part of frame 102 are lateral members 112. In FIG. 12, only certain ones of upstanding members 104, first and second upstanding members 106, 108, bracing members 110, and lateral members 112 have been numbered in order to maintain drawing clarity.

Further provided as a portion of frame 102 are cross-braces 114 desirably located at the top of pairs of second upstanding members 108 to increase lateral stability.

A given pair of first and second upstanding members 106, 108 can serve as parts of two adjacent upstanding portions 118 of frame 102 where frame 102 may comprise a number of such adjacent upstanding portions such as five such portions as illustrated in FIG. 12. Two such upstanding portions 118 are indicated and so-designated in FIG. 12.

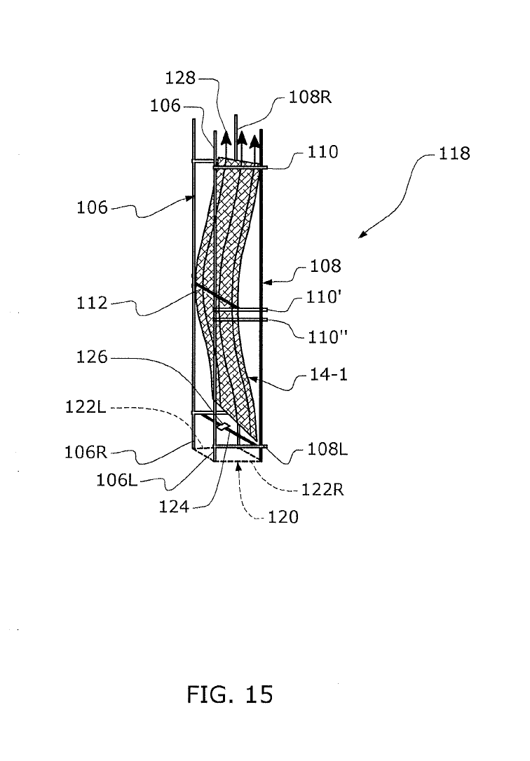

FIG. 15 illustrates, in vertically truncated form, a broken segment of one of upstanding portions 118. In FIG. 15, vertically upstanding members 106 and 108 are positioned at the corners of an imaginary rectangle, where the rectangle is illustrated in dotted lines and designated 120. The one of first upstanding members 106 at the left hand front side of the rectangle 120 is designated 106L in FIG. 15, while the one of first upstanding members 106 at the right hand side of rectangle 120 is designated 106R in FIG. 15. Similarly, toe one of second upstanding members 108 at the left hand side of rectangle 120 is designated 108L in FIG. 15 and the one of second upstanding members 108 located at the right hand side of rectangle 120 is designated 108R. Upstanding members 106L and 106R are considered to define the front of rectangle 120 where rectangle 120 is provided in this disclosure to clarify the geometry of the structure illustrated in FIG. 15.

There may optionally be provided first and second horizontally-oriented support members that are positionable on a floor or other surface to provide vertical support for upstanding portion 118 illustrated in FIG. 15; these optionally horizontally-oriented support members would run along the respective dotted lines designated 122L and 122R of rectangle 120 in FIG. 15.

As further illustrated in FIG. 15, a plurality of vertically-spaced apart parallel bracing members 110 connect respective ones of the upstanding first and second members 106, 108 along respective sides of rectangle 120. Bracing members 100 are preferably provided and oriented in closely vertically-spaced, adjacent pairs as illustrated by parallel bracing members 110′, 110″ in FIG. 15.

A plurality of lateral members 112 extend between and preferably slideably engage the vertically correspondingly positioned pairs 110′, 110″ of the horizontally-extending parallel bracing members 110. One such lateral member is indicated as 112 in FIG. 15. There is further provided a lateral member in the form of a cross-brace 114 at the top of each upstanding portion 118 where the cross-brace 114 is illustrated in FIG. 12.

Air permeable, antimicrobial, preferably botanically based, morbidity-inducing fabric, provided in the form of a strip 14-1 as illustrated in FIG. 15, is connected at the top of the strip either to an uppermost one of lateral members 112 or to fixed lateral bracing member 114. Fabric strip 14-1 extends downwardly as illustrated in FIG. 15 and may be positioned in various configurations by adjusting position of lateral members 112 with fabric strip 14-1 passing on a selected side of a given lateral member 112 thereby to provide the desired configuration for fabric strip 14-1. Specifically, lateral members 112 are moveably positionable along the pairs of parallel bracing members 110, between front and rear with respect to rectangle 120, to cause fabric portions 14-1 connected to the lateral members and extending between the lateral members to conform to selected contours. Desirably, a portion of the selected contour or all of the selected contour may approximate the upper surface of an air foil, in response to positioning of lateral members 112 and in response to air blowing thereagainst or therealong. Positioning of fabric strip 14-1 as the upper surface of an air foil facilitates generation of vortices along the air foil-like surface, thereby contributing to greater air flow through and along fabric strip 14-1, enhancing the antimicrobial, morbidity-inducing effect of fabric 14-1.

Optionally, a fixed horizontal brace illustrated as 124 may be provided at the bottom of FIG. 15 with a fan 126 mounted thereon to blow air upwardly against and along fabric strip 14-1 as indicated by arrows 128 at the top of FIG. 15.

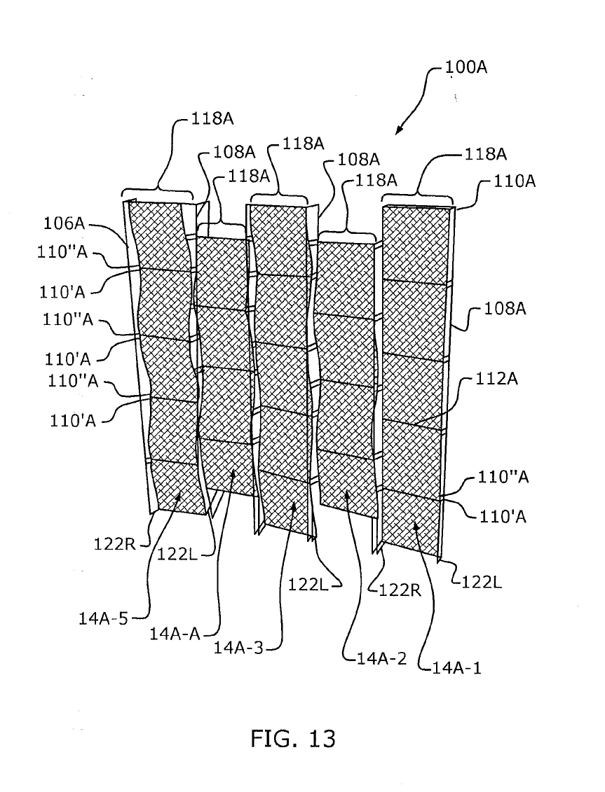

Referring to FIG. 13, the array 100A shown therein is similar to the array 100 illustrated in FIG. 12 and is constructed using segments as illustrated in FIG. 15. In FIG. 13, the upstanding portions 118 illustrated in FIG. 15 have been horizontally offset one from another front to back, relative to rectangles 120, thereby to provide a different and possibly more efficient configuration for array 100A. Other than the front to back offset of upstanding portions 118, array 100A in FIG. 13 is largely the same as array 100 illustrated in FIG. 12, as can be seen by comparing the drawings in which functionally equivalent and substantially corresponding parts have the same number, with the letter “A” used to distinguish parts illustrated in FIG. 13 from functionally identical or similar corresponding parts in FIG. 12.

With respect to array 100A illustrated in FIG. 13, a single first upstanding member 106A could not serve as support for adjacent upstanding portions 118A due to the horizontal offset of the upstanding portions 118A as illustrated in FIG. 13. However, a first upstanding member 106A of one upstanding portion 118A could serve as a second or rear upstanding member 108A of an adjacent upstanding portion 118A to horizontally offset as illustrated in FIG. 13.

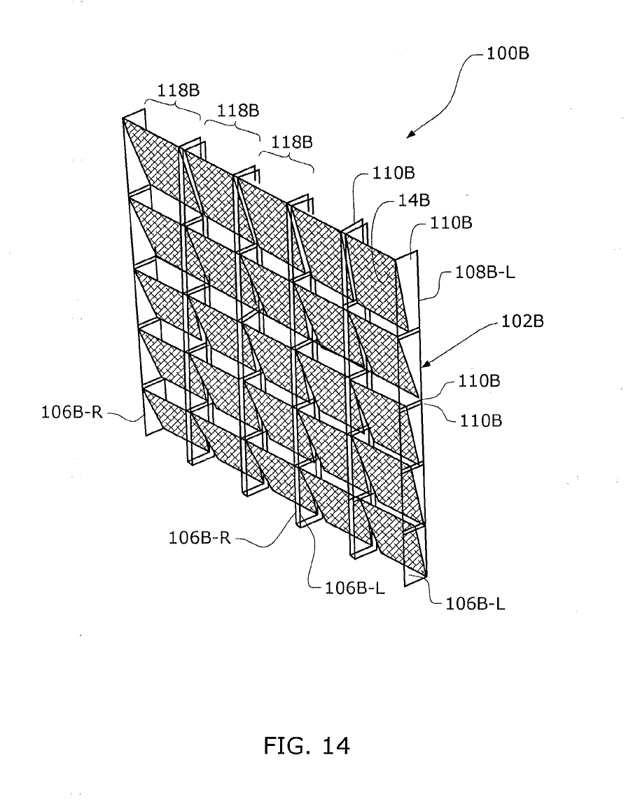

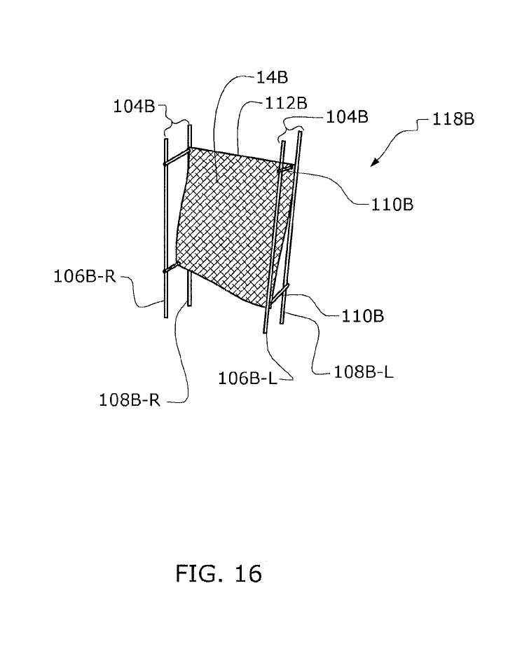

Referring to FIGS. 14 and 16, FIG. 14 illustrates another apparatus for improving indoor air quality in the form of an array 100B where array 100B includes a frame 102B that has vertically upstanding members 104B positioned at the corners of an imaginary rectangle with one edge of the rectangle being considered the front, in much the same manner as illustrated for FIGS. 12 and 15. Further similarly to FIGS. 12 and 15, one pair of upstanding members 104B has a first member 106B at the right front of the rectangle and a second member 108B at the right rear of the rectangle, and a second pair of upstanding members 104B having a first member at the left front of the rectangle and second member at the left rear of the rectangle where the members are designated 106B-L, 106B-R, 108B-L and 108B-R, with these designations being most clearly shown in FIG. 16. In array 100B illustrated in FIG. 14 and in FIG. 16, there are further provided a plurality of vertically-spaced apart bracing members 110B connecting respective ones of the upstanding first and second members 106B, 108B of the respective pairs of upstanding members 106B along respective sides of the imaginary rectangle. The imaginary rectangle is not illustrated in FIG. 14 nor in FIG. 16 to enhance drawing clarity.

As further illustrated in FIG. 14, the air permeable, antimicrobial, botanically based, morbidity-inducing fabric is not provided in the form of vertically elongated strips that extend from the top to the bottom of the apparatus 100B. Rather, the air permeable, antimicrobial, botanically based, morbidity-inducing fabric is provided in the form of rectangular sheets 14B where rectangular sheets 14B may be provided as several sheets, one above another, in each upstanding portion 118B of apparatus 100B. Fabric sheets 14B may be secured directly to bracing members 110B desirably by unnumbered rings fitting around bracing members 110B, thereby permitting movement of a fabric sheets 14B between forward upstanding members 106B-L and 106B-R and rear upstanding members 108B-L and 108B-R. Alternatively, lateral members 112B may be provided at either the top or the bottom or both of fabric sheet 14B with lateral members 112B desirably being movable between front and rear along bracing members 110B. With this arrangement, fabric sheets 14B can be adjusted to assume any of a plurality of configurations to take advantage of natural convention in the room in which array 100B is located.

One hundred percent (100%) cotton yarns and 100% cotton woven and knitted fabrics are desirably used to provide the air permeable, botanically based antimicrobial, morbidity inducing fabrics for the apparatus and practice of the methods of the invention. These fabrics are desirably treated using eugenol to impart antimicrobial microorganism morbidity-inducing properties to them.

Specifically, eugenol at 5 or 10 grams per liter may be mixed with polyvinyl alcohol at 5 or 10 grams per liter and 100 grams per liter glyoxal. The material to liquor ratio should be kept at either 1:10 or 1:20 by adding water to make up the difference between the liquor weight and that required by the liquor ratio calculation. The liquor ratio is calculated by weighing the fabric prior to treatment. The solution is desirably applied to the fabric by patting, such as by through the use of a Werner Mathis padder, and the fabric is then dried desirably in a through air oven at 80-85° C. for about four minutes. Thereafter, the fabric is cured, desirably in a through air oven, at a temperature ranging from 120-140° C. for from about three to about five minutes. With this approach, wet pick up, namely the amount of solution contained in the fabric after padding, amounts to about 65% of the weight of the dried fabric.

Another approach is to utilize instead a solution of eugenol and water, leaving out the polyvinyl alcohol and the glyoxal; the amount of eugenol may also be varied, as may be the liquor ratio and the wet pick up, with a suitable botanically based, antimicrobial, morbidity-inducing fabric resulting.

The fabric may also be made by knitting antimicrobial yarn into a 1×1 rib knit fabric. Plain knit fabric may also be used, but is not as tear-resistant as is rib knit fabric. Accordingly, plain knit fabric may be more difficult to install, whereas the heavier construction of the rib knit fabric eliminates many fabric tearing issues.

Fabrics, whether knit, woven or non-woven, made from different fiber types, including rayon, polyester, nylon and wool, may be used as the antimicrobial fabric.

Natural antimicrobials may be attached to the fabric using different methods. One additional method of attaching a natural antimicrobial botanically based, morbidity-inducing substance is to incorporate clove powder into polypropylene filaments during extrusion of the filaments, by mixing the clove powder with the polypropylene pellets to be extruded. This method incorporates the antimicrobial clove powder into the polypropylene filaments, which may then be knitted or woven into fabrics for use in the apparatus and methods of the invention. Using this method, mixing of the clove powder and the polypropylene pellets must be such that the mix is reasonably uniform. A non-uniform mix results in an undesirable inconsistent blend of clove powder and polypropylene along the length of the resulting polypropylene-clove filaments.

Applying natural botanically-based antimicrobials to other forms of fabrics, such as braided and non-woven, non-knit fabrics is also within the scope of the invention.

The eugenol approach described above for application of natural botanically based, antimicrobials to fabric may also be applied to yarns or fibers, since the fiber does not change its structure during fabric production processes.

Suitable antimicrobials have resulted using fabric knitted from yarns containing recycled fibers. The fiber content of these yarns was about 69% cotton, 29% acrylic, and 2% other materials. These recycled fibers had previously been dyed and had undergone several processing treatments as well as shredding during recycling. Reduction of SBS attained using these fabrics indicates that dyeing does not affect the efficacy of the treatment that produces antimicrobial properties in the fabric.

The following is claimed:

1) A modular unit for improving indoor air quality, comprising:
a) a frame surrounding an open interior and defining the outer periphery of the unit;
b) air permeable, antimicrobial, morbidity-inducing fabric, comprising a naturally occurring antimicrobial botanical compound, secured about the frame periphery on a first side of the frame and covering the open interior on a first side of the frame;
c) an air impermeable member secured about and covering the frame periphery on a remaining side of the frame;
d) at least one aperture formed in the frame and being adapted to house a fan therein;
e) a fan housed in the aperture for blowing air from outside the frame into the frame interior for subsequent passage of air blown into the frame interior outwardly through the fabric.

2) A modular unit for improving indoor air quality, comprising:
a) a frame having an open center and defining the outer periphery of the unit, the frame including a pair of parallel spaced apart lateral members connected by at least one tensioning cable and at least one aperture opening into the open center;
b) air permeable, antimicrobial, morbidity-inducing fabric comprising a naturally occurring antimicrobial botanical compound secured about the frame periphery on a first side of the frame and covering the open center;
c) the frame periphery on a remaining side of the frame being adapted to fit against a wall;
d) a fan mounted in the aperture for blowing air from outside the frame into the frame interior, for subsequent air passage outwardly through the fabric;
e) the cable(s) being adapted to connect to one or more cable gripping devices mounted in a wall so upon connection to the cable gripping devices the cable pulls the remaining side of the frame against the wall thereby closing the unit interior so that air may enter the unit interior only by action of the fan.

3) A modular unit for improving indoor air quality, comprising:
a) a frame surrounding an open interior and defining an outer periphery of the unit;
b) air permeable, antimicrobial, morbidity-inducing fabric comprising a naturally occurring antimicrobial botanical compound secured about the periphery on a first side of the frame and covering the open interior on the first side of the frame;
c) at least one aperture formed in the frame;
d) a fan mounted in the aperture for blowing air from outside the frame into the frame interior, for subsequent air passage outwardly through the fabric;
e) the unit being mountable against a vertical wall by a portion of the frame hanging on at least one member protruding from the wall, with a second side of the frame being flush with the wall so that air blown into the open interior of the frame by the fan escapes from the frame interior by passing through the fabric.

4) A modular unit for improving indoor air quality, comprising:
a) a frame surrounding an open interior and defining the outer periphery of the unit;
b) air permeable, antimicrobial, morbidity-inducing fabric comprising a naturally occurring antimicrobial botanical compound secured about the frame periphery on a first side of the frame and covering the open interior on the first side of the frame;
c) a pair of apertures each formed in the frame, one aperture in an upper extremity of the frame and the other aperture in a lower extremity of the frame;
d) at least one fan mounted on the frame and extending into the open interior, aligned with one of the apertures, for drawing air from outside the frame into the frame interior through the aligned aperture, for subsequent air passage from the frame interior outwardly through the fabric;
e) the frame being mountable against a vertically extending wall by an upper portion of the frame hanging on at least one member protruding from the wall, with a second side of the frame being substantially flush with the wall so that air drawn into the open interior of the frame by the fan escapes by passing through the fabric.

5) A modular unit for improving indoor air quality, comprising:
a) a horizontal frame surrounding an open interior and defining a portion of the outer periphery of a filter portion of the unit having an outwardly facing upwardly elongated surface;
b) air permeable, antimicrobial, morbidity-inducing fabric comprising a naturally occurring antimicrobial botanical compound secured around the frame periphery on a downwardly facing side of the frame and covering the open interior on the downwardly facing side of the frame;
c) porous light-reflective fabric secured about the frame periphery on the upwardly facing side of the frame and covering the frame periphery on the upwardly facing side of the frame;
d) at least one fan mounted in the frame for blowing air from outside the frame into the frame interior, for subsequent passage of air blown into the frame interior, outwardly through the air permeable fabric.
e) an exterior portion of the frame outer periphery being planar for facingly contacting a portion of a window;
f) at least one member extending upwardly from the frame for upwardly supporting the frame about a pivotal connection at a position remote from the planar portion of the frame to permit weight of the frame to urge the frame against the portion of the window and maintain the frame in a horizontal plane;
g) at least one solar cell positionable to receive solar energy through the window, connected to and powering the fan.

6) A modular unit for improving indoor air quality, comprising:
a) a frame surrounding an open interior and defining the outer periphery of the unit;
b) air permeable, antimicrobial, morbidity-inducing fabric comprising a naturally occurring antimicrobial botanical compound secured about the frame periphery on an upper side of the frame and covering the open interior on the upper side of the frame;
c) porous reflective non-woven fabric secured about the frame periphery on the remaining side of the frame and covering the frame periphery on the remaining side of the frame;
d) at least one aperture formed in the frame being adapted to house a fan therein;
e) a fan mounted in the aperture for blowing air into the frame interior for subsequent passage of that air outwardly through the air permeable fabric.
f) struts extending from the frame, for supporting the unit by suspension thereof from the struts in mid-air within a room in a building.

7) Apparatus for combating sick building syndrome, comprising:
a) a plenum at least partially bounded by fabric comprising at least one naturally occurring anti-bacterial substance exhibiting microorganism morbidity inducing properties; and
b) a fan for introducing air into the plenum for passage outwardly through the fabric.

8) Apparatus for combating sick building syndrome, comprising:
a) fabric comprising at least one naturally occurring substance exhibiting microorganism morbidity inducing properties; and
b) a frame adapted for affixation of the fabric thereacross; and
c) a fan for blowing air through a portion of the fabric affixed across the frame.

9) Apparatus of claim 7 wherein the naturally occurring anti-bacterial substance is botanically based.

10) A method for combating sick building syndrome, comprising:
a) providing a plenum that is at least partially bounded by fabric including at least one naturally occurring substance exhibiting microorganism morbidity inducing properties; and
b) introducing air into the plenum for passage outwardly through the fabric.

11) A method for combating sick building syndrome, comprising:
a) providing a fabric comprising at least one naturally occurring substance exhibiting microorganism morbidity inducing properties, affixed across a frame; and
b) blowing air through a portion of the fabric affixed across the frame.

12) The method of claim 11 wherein the naturally occurring substance exhibiting microorganism morbidity inducing properties is clove oil.

13) The method of claim 11 wherein the naturally occurring substance exhibiting microorganism morbidity inducing properties is eugenol.

14) Apparatus of claim 7 wherein the naturally occurring substance exhibiting microorganism morbidity inducing properties is clove oil.

15) Apparatus of claim 7 wherein the naturally occurring substance exhibiting microorganism morbidity inducing properties is eugenol.

16) Apparatus for improving indoor air quality, comprising:
a) vertically upstanding members positioned at the corners of an imaginary rectangle, one edge of the rectangle being considered the front;
b) one pair of members having a first member at the right front of the rectangle and the second member at the right rear of the rectangle and a second pair of members having a first member at the left front of the rectangle and a second member at the left rear of the rectangle;
c) a plurality of vertically spaced apart bracing members connecting respective ones of the upstanding first and second members of the respective pairs of upstanding members along respective sides of the rectangle;
d) lateral members extending between vertically correspondingly positioned pairs of the horizontally extending parallel bracing members;
e) air permeable, botanically based antimicrobial, morbidity-inducing fabric connected to and extending between vertically adjacent pairs of lateral members;
f) the lateral members being movably positionable along the parallel bracing members, between front and rear, to cause the fabric portions connected thereto and extending therebetween to conform to selected contours, a portion of or all of which may approximate the upper surface of an airfoil, in response to air blowing thereagainst.

17) Apparatus for improving indoor air quality, comprising:
a) vertically upstanding members positioned at the corners of an imaginary rectangle, one edge of the rectangle being considered the front;
b) one pair of members having a first member at the right front of the rectangle and the second member at the right rear of the rectangle and a second pair of members having a first member at the left front of the rectangle and a second member at the left rear of the rectangle;
c) a plurality of vertically spaced apart bracing members connecting respective ones of the upstanding first and second members of the respective pairs of upstanding members along respective sides of the rectangle;
d) air permeable, botanically based antimicrobial, morbidity-inducing fabric connected to and extending between the vertically spaced apart bracing members.
